(19) United States
(12) Patent Application Publication
(10) Pub. No.: US 2026/0009795 A1
(43) Pub. Date: Jan. 8, 2026

(54) LYMPHOCYTE SELECTION METHOD

(71) Applicants: PEPTINOV, Paris (FR); CONSERVATOIRE NATIONAL DES ARTS ET METIERS, Paris (FR); INSTITUT PASTEUR, Paris (FR)

(72) Inventors: Gabriel MOREAU, HOUSSAY (FR); Matthieu SCHOENHALS, MONTPELLIER (FR); Jean-Francois ZAGURY, LE VESINET (FR)

(21) Appl. No.: 18/863,451

(22) PCT Filed: May 6, 2023
(86) PCT No.: PCT/EP2023/062074

§ 371 Date: Nov. 6, 2024

(30) Foreign Application Priority Data
May 6, 2022 (FR) FR2204322

(51) Int. Cl. G01N 33/569; C07K 16/00; C12N 5/00; C12N 5/0781; C12N 5/0783; G01N 15/149; G01N 33/50; G01N 33/543; G01N 33/68

(52) U.S. Cl. CPC G01N 33/56983; C07K 16/00; C12N 5/0087; C12N 5/0635; C12N 5/0636; G01N 15/149; G01N 33/5005; G01N 33/54366; G01N 33/6854; G01N 2474/10

(57) ABSTRACT

The present invention relates to a method for selecting lymphocytes recognizing a conformational epitope of a protein antigen from a population of cells, comprising a step of identifying at least one lymphocyte binding to at least two separate peptides comprising sequences of the protein antigen, in particular at least three separate peptides comprising sequences of the protein antigen.

### FIELD OF THE INVENTION

The present invention relates to a method for selecting lymphocytes, in particular B or T lymphocytes, as well as a method for preparing antibodies from the selected B lymphocytes.

### TECHNICAL BACKGROUND

Interest in therapeutic monoclonal antibodies continues to grow, year after year. Indeed, their highly specific affinity for antigens makes it possible to provide highly effective medical treatments. As such, between 2005 and 2017, the number of monoclonal antibodies approved by the U.S. Food and Drug Administration rose from 2 to 64.

However, there remains a critical hurdle to overcome in preparing effective monoclonal antibodies.

Indeed, the selection of B lymphocytes from which monoclonal antibodies are derived is often based on the primary structure of the target protein antigens, i.e. the linear epitopes to which the antibodies bind, whereas it is epitopes derived from the three-dimensional, tertiary or quaternary, structure of the proteins that most often provide the best affinity for the antibodies and thus best ensure their biological functions.

Thus, making it possible to obtain monoclonal antibodies specific to conformational epitopes, thus recognizing the three-dimensional structure of target antigens, should improve the avidity of these antibodies, and hence their use as experimental tools, diagnostic markers or immunotherapeutics.

In this context, Tsumoto et al (2019) Immunotherapy 11:119-127 propose to prepare monoclonal antibodies directed against conformational epitopes of an antigen by immunizing mice with DNA molecules encoding the antigen, isolating B lymphocytes and then fusing them with myeloma cells expressing this antigen to obtain hybridomas producing monoclonal antibodies specific for the three-dimensional structure of the antigen.

However, while this process does produce monoclonal antibodies directed against conformational epitopes, it is relatively complex to implement.

Therefore, it still remains to provide an easy-to-implement method for obtaining monoclonal antibodies directed against conformational epitopes.

### SUMMARY OF THE INVENTION

The present invention stems from the inventors' unexpected finding that it was possible to select B lymphocytes specific for a conformational epitope of an antigen using two distinct peptides derived from the antigen and three-dimensionally close in the structure of the antigen.

Thus, the present invention concerns a method for selecting lymphocytes, in particular B or T lymphocytes, recognizing a protein antigen, in particular a conformational epitope of a protein antigen, from a population of cells, comprising a step of identifying at least one lymphocyte, in particular B or T lymphocyte, binding to at least two distinct peptides comprising sequences derived from the protein antigen, in particular at least three distinct peptides comprising sequences derived from the protein antigen.

The present invention also relates to a method for preparing at least one antibody, or antibody fragment, directed against a protein antigen, wherein the antibody, or antibody fragment, is prepared from at least one B lymphocyte obtained by carrying out the method for selecting B lymphocyte as defined above.

The present invention also relates to a method for preparing at least one TCR, or TCR fragment, directed against a protein antigen, wherein the TCR, or TCR fragment, is prepared from at least one T lymphocyte obtained by carrying out the method for selecting T lymphocyte as defined above.

The present invention also relates to antibodies, or antibody fragments, and TCRs, or TCR fragments, obtainable by implementing the methods of preparation according to the invention.

The present invention also relates to antibodies, or antibody fragments, and TCRs, or TCR fragments, obtainable by implementing the methods of preparation according to the invention for their use as drugs or diagnostic agents.

The present invention also relates to the in vitro use of antibodies, or antibody fragments, and TCRs, or TCR fragments, obtainable by implementing the methods of preparation according to the invention for experimental or diagnostic purposes.

### DETAILED DESCRIPTION OF THE INVENTION

As used here, the term “comprising” is synonymous with “including”, “containing” or “encompassing”, i.e. when an object “comprises” one or more features, features other than those mentioned may also be included in the object. Conversely, the expression “consisting of” means “constituted by”, i.e. when an object “consists of” one or more features, the object cannot include features other than those mentioned.

### Lymphocytes

Lymphocytes according to the invention are preferably B lymphocytes and/or T lymphocytes. By “B lymphocyte” we mean any cell of the B lineage, such as a naive B lymphocyte, an activated B lymphocyte, a memory B lymphocyte, a plasmablast or a plasma cell, in particular a long-lived plasma cell. A T lymphocyte is any cell of the T lineage.

As will become clear to those in the trade, B lymphocytes recognize the protein antigen via the antibodies they carry, and T lymphocytes recognize the protein antigen via the T cell receptor (TCR).

### Cells

The cell population is of any type likely to include lymphocytes, particularly B or T lymphocytes.

The cell population may come from a single individual or from several individuals. Preferably, the cell population is obtained from a biological sample or specimen of one or more individuals, such as a whole blood or bone marrow sample or specimen. The individual is preferably a human or an animal, in particular a mammal. The individual is preferably immunized against the protein antigen, which may be of the same species or of a different species from that of the individual, in particular an infectious agent. Immunization may be natural, for example when the protein antigen is derived from an infectious agent that has previously infected the individual, or it may be induced by administration to the individual of the protein antigen or part thereof, or of a nucleic acid, DNA or RNA, encoding the protein antigen or part thereof, i.e. by active immunization or vaccination.

Preferably, the cell population is a population of peripheral blood mononuclear cells (PBMCs).

Preferably, the method for identifying lymphocytes, in particular B or T lymphocytes, as defined above, comprises a prior step of selecting lymphocytes, in particular B or T lymphocytes, from the cell population. As understood herein, the prior step of selecting lymphocytes, in particular B or T lymphocytes, may result in a subpopulation comprising only lymphocytes, in particular B or T lymphocytes, or enriched in lymphocytes, in particular B or T lymphocytes. This selection can be carried out using ligands, in particular antibodies, targeting membrane markers specific to lymphocytes, in particular B or T lymphocytes, and the antibodies can be coupled to magnetic beads or to luminophores, in particular fluorophores, to facilitate the detection, selection, isolation or purification of lymphocyte-antibody complexes.

### Peptides

As understood here, protein antigen-derived sequences are portions of contiguous amino acid residue sequences of the total amino acid residue sequence of the protein antigen.

Preferably, the at least two distinct peptides respectively comprise from 6 to 30 amino acid residues, 6 to 25 amino acid residues, 6 to 20 amino acid residues, 6 to 15 amino acid residues, 10 to 30 amino acid residues, 10 to 20 amino acid residues, 12 to 20 amino acid residues, 10 to 18 amino acid residues, or 10 to 15 amino acid residues.

Preferably, the at least two distinct peptides comprise at least 6, 7, 8, 9, 10, 11 or 12 amino acid residues respectively.

Preferably, the at least two distinct peptides comprise not more than 30, 25, 20 or 15 amino acid residues respectively.

Preferably, the at least two peptides comprise non-overlapping sequences from the protein antigen. As understood here, non-overlapping sequences are such that they do not overlap within the primary structure of the antigen, i.e. they are disjoint peptides.

Preferably, the at least two peptides are at a distance from each other of 3.10⁻⁹ m, 2.5.10⁻⁹, 2.10⁻⁹ m, 10⁻⁹ m or less in the three-dimensional structure of the protein antigen.

The three-dimensional structure of the protein antigen can be obtained by X-ray crystallography, nuclear magnetic resonance (NMR), microscopy or computerized three-dimensional structure prediction.

The distance between the at least two distinct peptides can be calculated in many ways well known to those skilled in the art. It can be the average of the distances between each amino acid residue of one of the peptides to each amino acid residue of the other peptide(s). Preferably, it is the minimum distance among the respective distances between each amino acid residue of one of the peptides to each amino acid residue of the other peptide(s).

As understood here, the distance between two amino acid residues is the distance between the respective alpha-carbon centers of each residue.

### Protein Antigen

The protein antigen according to the invention can be a protein or protein complex of any type. In particular, it can be a monomeric or multimeric protein, especially a homomultimeric or heteromultimeric protein. In an embodiment of the invention, particularly in the context of a method of selecting T lymphocytes as defined above, the protein antigen is a complex between a peptide, a polypeptide or an antigenic protein and an antigen-presenting protein, in particular a major histocompatibility complex (MHC) molecule.

Preferably, the protein antigen comprises at most 5000, 2500, 1000 or 500 amino acid residues.

Preferably, the protein antigen comprises at least 25, 50, 75, 100 or 200 amino acid residues.

Preferably, the protein antigen comprises from 25 to 5000, more preferably from 50 to 2500, even more preferably from 75 to 2000 amino acid residues.

The protein antigen may consist solely of amino acid residues or include aprotic components in addition to amino acid residues. The protein may be substituted by at least one polysaccharide and/or at least one lipid. In addition, the protein may comprise one or more prosthetic groups, such as a heme group, a cofactor, a nucleic acid or an iron-sulfur cluster.

The protein antigen can be derived from any type of organism. Preferably, the protein antigen is derived from an infectious agent or from a human or animal protein.

In particular, the protein antigen can be obtained by purification from the organism that produces it naturally, or recombinantly, from cell cultures of any type (eukaryotic or prokaryotic).

### Identification

As will be clear for the person skilled in the art, the step of identifying at least one lymphocyte, in particular a B or T lymphocyte, that binds to at least two distinct peptides comprising sequences derived from the protein antigen comprises contacting the peptides with the cell population to identify within the cell population at least one lymphocyte that binds to the at least two distinct peptides.

Preferably, the method of selection as defined above includes an additional step of isolating at least one lymphocyte, in particular a B or T lymphocyte, which binds to the at least two distinct peptides of the protein antigen.

The identification, and possibly the isolation, of lymphocytes, particularly B or T lymphocytes, binding to at least two distinct peptides can be achieved by numerous techniques well known to those skilled in the art. Examples include successive passage of cells over chromatographic columns on which the peptides are fixed (a first peptide then selection, a second peptide then selection), or successive binding/selection with magnetic beads carrying the peptides (a first peptide then selection, a second peptide then selection), or successive attachment/selection to plates displaying the peptides, or cell sorting combined with flow cytometry where the cells are first identified and then sorted. In the case of B lymphocytes, it is also possible to clone cells by limiting dilution and to test antibodies in the supernatant that recognize both of the two or more peptides. Once the lymphocytes have been selected and cloned for recognition of at least two distinct peptides, their variable sequences can be sequenced directly or after amplification of the selected clone.

Preferably, the identification, and optionally isolation, step is carried out by flow cytometry, with optionally cell sorting.

When the identification, and optionally isolation, step is carried out by flow cytometry, the at least two distinct peptides are preferably labeled with distinct luminophores, in particular fluorophores. This ensures that the distinct peptides are recognized by the same lymphocyte, in particular B or T lymphocyte.

Furthermore, when the identification, and optionally isolation, step is carried out by flow cytometry, the identification of peptides binding to the lymphocyte can be carried out by contacting the cell population with the distinct peptides added all at the same time, i.e. during the same sorting, or sequentially, peptide by peptide, i.e. in several successive sortings, e.g. by isolating lymphocytes using a first peptide, then using a second peptide on the lymphocytes sorted with the first peptide, then possibly using one or more additional peptides on the lymphocytes sorted with the first two peptides etc., Contacting the cell population with the peptides added at the same time will generally require each peptide to be labelled with a different marker. In contrast, contacting the cell population with sequentially added peptides does not require each peptide to be labeled with a different marker, and may even allow only one type of peptide label to be used.

Preferably, the method for selecting lymphocytes, in particular B lymphocytes, recognizing a conformational epitope of a protein antigen, from a population of cells according to the invention, comprises:
a step of selecting lymphocytes from the population of cells;
a step of identifying at least one lymphocyte, in particular a B lymphocyte, binding to at least two distinct peptides, in particular of 6 to 30 amino acid residues, comprising sequences, in particular non-overlapping sequences, derived from the protein antigen, the at least two distinct peptides being in particular at a distance from each other of 3.10-9 m or less in the three-dimensional structure of the protein antigen;
a step of isolating at least one lymphocyte identified in the previous step.

### Antibodies-TCR

As used herein, the term “antibody” encompasses antibodies in their entirety as well as antibody fragments comprising at least one antigen-binding portion, such as V_{L} and/or V_{H}, Fab, F(ab′)₂, and scFv fragments. The antibody according to the invention may be derived from a single species, be chimeric, humanized or human. The antibody can be monospecific or bispecific. Furthermore, the antibody according to the invention may be monomeric or multimeric, in particular dimeric or pentameric. The antibody according to the invention may be of isotype A, D, E, G or M, preferably G.

The antibody, or TCR, directed against a protein antigen is preferably an antibody, or TCR, recognizing a conformational epitope of the protein antigen.

The antibody, or TCR, directed against a protein antigen is preferably an antibody, or TCR, specific to a three-dimensional structure of the protein antigen.

The antibody directed against a protein antigen is preferably a monoclonal antibody.

The antibody directed against a protein antigen can be prepared from a purified B lymphocyte, using a number of techniques well known to the person skilled in the art.

By way of example, the B lymphocyte, optionally after clonal multiplication, can be fused with a myeloma cell to yield a monoclonal antibody-producing hybridoma. By way of example also, the portions of the B lymphocyte genome coding for all or part of the antibody, in particular its variable parts, can be cloned and then recombinantly expressed by cultured cells, possibly after insertion in a chimeric, humanized or human antibody structure.

The TCR directed against a protein antigen can be prepared from a T lymphocyte by numerous techniques well known to the person skilled in the art.

By way of example, the portions of the T lymphocyte genome coding for all or part of the TCR, in particular its variable parts, can be cloned and then recombinantly expressed by other cells. In particular, TCRs can be produced in the form of fused chimeric proteins to stabilize them and facilitate their use, for example with constant parts of immunoglobulins.

The invention is further explained with the aid of the following non-limiting examples and figures.

### DESCRIPTION OF FIGURES

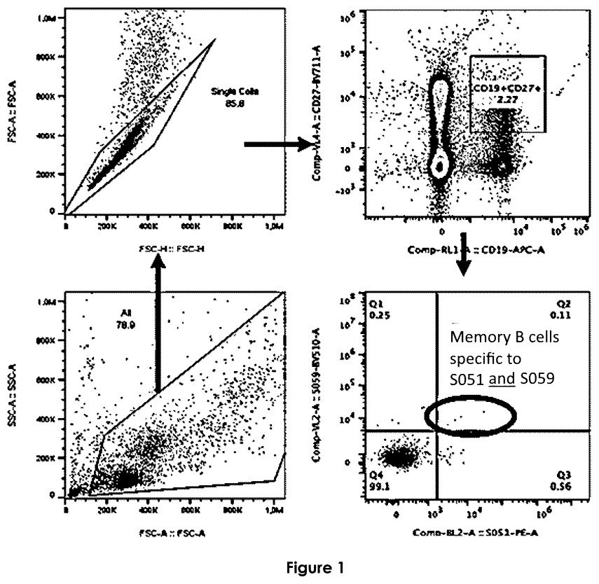

FIG. 1 shows four flow cytometry graphs (panels) representing events sorted:
as a function of diffracted light measured in front of the cytometer's laser beam (parameter FSC-A::FSC-A) and measured from the side (parameter SSC-A::SSC-A) (bottom left panel),
as a function of the diffracted light measured in front of the cytometer laser beam (parameters FSC-H::FSC-H/FSC-A::FSC-A) (top left panel),
as a function of light emitted by the fluorophores allophycocyanin (APC) and BV711™ (parameters comp-VL4-A::CD27-BV711-A/comp-RL1-A::CD19-APC-A) (top right panel),
as a function of light emitted by BV510™ and PE fluorophores (parameters comp-VL2-A::S059-BV510-A/comp-BL2-A::S051-PE-A) (bottom right panel).

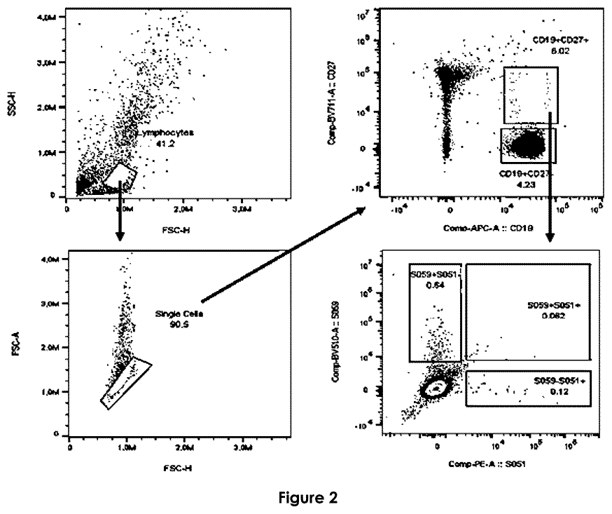

FIG. 2 shows four flow cytometry graphs (panels) representing events sorted:
as a function of diffracted light measured in front of the cytometer's laser beam (parameter FSC-H) and measured from the side (parameter SSC-H) (top left panel),
as a function of the diffracted light measured in front of the cytometer's laser beam (FSC-A and FSC-H parameters) (bottom left panel),
as a function of light emitted by the fluorophores allophycocyanin (APC) and BV711™ (parameters Comp-BV711-A::CD27-/Comp-APC-A::CD19) (top right panel),
as a function of light emitted by BV510™ and PE fluorophores (parameters Comp-BV510-A::S059-/Comp-PE-A::S051-) (bottom right panel).

### EXAMPLES

### Example 1

Peripheral blood mononuclear cells (PBMC) from an individual immunized against Covid-19 were purified by Ficoll.

The cells were then labeled using the following antibodies: anti-CD19-APC, anti-CD27-BV711, which mark memory B cells.

Next, cells were labeled with the two biotinylated peptides S051 and S059 following the recommendations from the supplier Thermo Scientific™ (EZ-Link™ Sulfo-NHS-LC-Biotinilation kit) and coupled to fluorescent Streptavidin-BV510™ and Streptavidin-phycoerythrin (PE) conjugates. The S051 peptide consists of residues 201 to 215 of the SARS-COV-2 Spike protein, and the S059 peptide consists of residues 233 to 247 of the SARS-COV-2 Spike protein. Peptides S051 and S059 are disjoint (non-overlapping) in the Spike protein sequence, but neighboring three-dimensionally in that they are located within 25 Angstroms of each other in the three-dimensional structure of the Spike protein.

The inventors analyzed 231,000 events by flow cytometry, from which they visually removed cell-free debris (bottom left panel of FIG. 1), then eliminated doublets (top left panel of FIG. 1). They then selected the CD19⁺ CD27⁺ events (top right panel of FIG. 1) corresponding to memory B lymphocytes. Among these cells, 4 have antibodies on their surface with affinity for both S051 and S059 (bottom right panel of FIG. 1).

Statistics for the CD19+/CD27+ population are summarized in Table 1 below:

TABLE 1

| | CD19+/ | S051+/ | S051−/ | S051+/ | S051−/ |
|---|---|---|---|---|---|
| | CD27+ | S059+ | S059+ | S059− | S059− |
| | cells | cells | cells | cells | cells |
| Quantity | 3554 | 4 | 9 | 20 | 3521 |
| % among | — | 0.11 | 0.25 | 0.56 | 99.1 |
| CD19+/CD27+ | | | | | |
| cells | | | | | |
| % among PBMC | 2.27 | 1.73 · 10⁻³ | 3.89 · 10⁻³ | 8.64 · 10⁻³ | 1.52 |

The inventors were thus able to identify and isolate four distinct B lymphocytes recognizing both the distinct, non-overlapping S051 and S059 peptides. This indicates that the identified B cells carry antibodies recognizing both the S051 peptide and the S059 peptide, which are disjoint in the primary Spike structure. These antibodies therefore recognize a conformational epitope of the SARS-COV-2 Spike protein.

### Example 2

### 1. Sorting of Specific Memory B Lymphocytes and Culture of Clones.

Two 8 mL BD Vacutainer® CPTT citrated tubes were collected from a healthy individual who had undergone a full vaccination regimen and 2 SARS-CoV-2 infections. These tubes allow very simple separation of peripheral blood mononuclear cells (PBMC). The purified PBMCs were then labeled with 2 fluorophore-coupled peptides, S051 and S059, identified in Example 1 as part of a conformational epitope of the Spike protein. These two peptides, S051 and S059, were coupled to phycoerythrin (PE) and brilliant violet fluorochrome 510 (BV510™) respectively, as shown in Example 1.

Labeling of memory B cells was performed with labeled antibodies (anti-CD19-APC, anti-CD27-BV711™).

Sorting was then carried out on a flow cytometry platform (Cytek Aurora cell sorter) as shown in FIG. 2, and in the end, forty-eight single cells labeled with all fluorophores (PE+, BV510+, APC+, BV711+) were sorted on cell sheets of Feeder MS5-CD40L cells previously spread on 96-well flat-bottom plates.

Media were changed 2 times a week for 4 weeks. (RPMI-1640 supplemented with 10% FCS, 55 UM 2-ME, 1% Pen Strep (100 units/ml penicillin, 100 g/ml streptomycin), 10 mM HEPES, 1 mM sodium pyruvate and 1% MEM NEAA with recombinant human IL-2 (Peprotech 200-02, 500 μg; final culture concentration 50 ng/ml), recombinant human IL-4 (Peprotech 200-04, 100 μg; final concentration 10 ng/ml), recombinant human IL-21 (Peprotech 200-21, 100 μg; final concentration 10 ng/ml), and recombinant human BAFF (Peprotech 310-13, 100 μg; final concentration 10 ng/ml).

Supernatants were collected and frozen at −20° C. before being tested for the presence of antibodies.

### B. Test of Supernatants for the Presence of Specific IgG.

The thus prepared supernatants were tested for the presence of IgG by ELISA. Positive wells were then tested by Western blot for the presence of IgG specific to the Spike protein. The presence of IgG is highlighted by an anti-IgG-HRP antibody and the optical density measured by luminescence.

30 supernatants tested from the 48 clones initially sorted showed variable levels of antibody, 7 of them at high levels (ranging from 4 to 15 luminescence units). 2 of these 7 supernatants responded positively for the presence of IgG specific to recombinant Spike protein deposited on nitrocellulose membrane.

This shows that sorting memory B lymphocytes by flow cytometry using the 2 fluorescently-labeled peptides does indeed yield monoclonal antibodies recognizing a conformational epitope within the Spike protein.

In addition, it is verified that these supernatants also recognize each of the two isolated peptides.

1-12. (canceled)

13. A method for selecting lymphocytes recognizing a conformational epitope of a protein antigen from a population of cells, comprising a step of identifying at least one lymphocyte binding to at least two distinct peptides comprising sequences derived from the protein antigen.

14. The method of selection of claim 13, comprising a prior step of selecting lymphocytes from the cell population.

15. The method of selection of claim 13, wherein the lymphocytes are B lymphocytes or T lymphocytes.

16. The method of selection of claim 13, wherein the lymphocytes are B lymphocytes.

17. The method of selection of claim 13, wherein the cell population is a peripheral blood mononuclear cell (PBMC) population.

18. The method of selection of claim 13, wherein the at least two distinct peptides respectively comprise from 6 to 30 amino acid residues.

19. The method of selection of claim 13, wherein the at least two distinct peptides comprise non-overlapping sequences derived from the protein antigen.

20. The method of selection of claim 13, wherein the at least two distinct peptides are at a distance from each other of 3.10-9 m or less in the three-dimensional structure of the protein antigen.

21. The method of selection of claim 13, comprising an additional step of isolating at least one lymphocyte binding to the at least two distinct peptides.

22. The method of selection of claim 13, wherein the identification, and optionally isolation, step is carried out by flow cytometry, optionally with cell sorting.

23. The method of selection of claim 13, wherein the protein antigen is derived from an infectious agent or from a human or animal protein.

24. A method for preparing at least one antibody, or antibody fragment, directed against a protein antigen, wherein the antibody, or antibody fragment, is prepared from at least one B lymphocyte obtained by carrying out the lymphocyte selection process as defined in claim 13.
